(19) United States
(12) Patent Application Publication
(10) Pub. No.: US 2022/0283110 A1
(43) Pub. Date: Sep. 8, 2022

(54) DEVICE, TUNNEL CURRENT MEASURING APPARATUS, NUCLEIC ACID SEQUENCE READING APPARATUS, TUNNEL CURRENT MEASURING METHOD, AND NUCLEIC ACID SEQUENCE READING METHOD

(71) Applicant: OSAKA UNIVERSITY, Osaka (JP)

(72) Inventors: Takahito OHSHIRO, Osaka (JP); Masateru TANIGUCHI, Osaka (JP); Yuki KOMOTO, Osaka (JP)

(21) Appl. No.: 17/686,249

(22) Filed: Mar. 3, 2022

(30) Foreign Application Priority Data
Mar. 4, 2021 (JP) 2021-034256

(51) Int. Cl. G01N 27/327; G01N 27/447

(52) U.S. Cl. CPC G01N 27/3278; G01N 27/44791; B82Y 40/00

(57) ABSTRACT

Provided is a device that facilitates a sample to enter a sample measurement channel in which measuring electrodes are arranged. A device used in measurement of tunnel current includes: a base material; a channel formed in the base material; and a pair of measuring electrodes for measuring tunnel current occurring when a sample passes between the pair of measuring electrodes. The channel includes a sample supply channel, a sample measurement channel in which the measuring electrodes are arranged, a first taper channel arranged between the sample supply channel and the sample measurement channel and having a channel width that decreases from the sample supply channel to the sample measurement channel, and a sample collection channel used for collecting a sample that passed through the sample measurement channel. The width of a connection part between the first taper channel and the sample measurement channel is 20 nm to 200 nm.

### CROSS-REFERENCE TO RELATED APPLICATION

The present application is based on and claims priority from Japanese Patent Application No. 2021-034256, filed Mar. 4, 2021, the disclosure of which is hereby incorporated by reference herein in its entirety.

### BACKGROUND OF THE INVENTION

### Field of the Invention

The disclosure in the present application relates to a device, a tunnel current measuring apparatus, a nucleic acid sequence reading apparatus, a tunnel current measuring method, and a nucleic acid sequence reading method.

### Description of the Related Art

Memories using DNA that is a biopolymer are paid attention for their high storage density and storage retention stability. Commercialization of DNA memories requires a technology to read a DNA sequence at a high rate. As a method of reading a DNA sequence, a known exemplary method is to optically detect an elongation reaction in a PCR amplification process by using an optical probe. Devices used for such a method are called a sequencer and have already been commercialized. However, since such a method requires an amplification process such as a PCR, there is a problem of inability of achieving a reading rate exceeding an elongation time (about one second per one base) due to the amplification process. Further, since a PCR is effective only for DNA and thus is not applicable to biomolecules, artificial bases, and the like other than DNA, there also is a problem of a limited storage density that can be achieved with storage in combination of only four types of bases.

As a nucleic acid reading apparatus (method) other than the PCR, there is a method of forming a nanopore (micro through hole) in a thin film and measuring tunnel current when a nucleic acid passes through the nanopore (see Japanese Patent Application Laid-Open No. 2017-509899).

Further, it is desirable to elongate a nucleic acid when reading the nucleic acid. As a technology to elongate a nucleic acid, for example, a technology to provide a base material with a channel having nanowires formed therein and pass a nucleic acid through the spacing between the nanowires to be elongated (see Japanese Patent Application Laid-Open No. 2016-103979) is also known.

It is desirable that a reading apparatus (reading method) of a DNA memory can improve the reading rate of a nucleic acid and read a nucleic acid sequence even with a small amount of a sample. In the reading apparatus (reading method) disclosed in Japanese Patent Application Laid-Open No. 2017-509899, a chamber is formed by a thin film as a boundary in which a nanopore is formed, and tunnel current occurring when a nucleic acid supplied into the chamber passes through the nanopore is measured. However, the opening of the nanopore is formed so as to be substantially perpendicular to the thin film, and the area of the opening to the thin film is significantly small. Thus, a sample liquid containing nucleic acids comes into contact with the entire thin film, and this causes a problem that the nucleic acids in the sample liquid have difficulty in entering the opening.

### SUMMARY OF THE INVENTION

An object of the disclosure of the present application is to provide a device that solves the above problem, a tunnel current measuring apparatus and a tunnel current measuring method using the device, and a nucleic acid sequence reading apparatus and a nucleic acid sequence reading method.

The disclosure of the present application relates to a device, a tunnel current measuring apparatus, a nucleic acid sequence reading apparatus, a tunnel current measuring method, and a nucleic acid sequence reading method illustrated below.

(1) A device used in measurement of tunnel current, the device including:

a base material;

a channel formed in the base material; and

a pair of measuring electrodes for measuring tunnel current occurring when a sample passes between the pair of measuring electrodes,

wherein the channel includes

a sample supply channel,

a sample measurement channel in which the measuring electrodes are arranged,

a first taper channel arranged between the sample supply channel and the sample measurement channel and having a channel width that decreases from the sample supply channel to the sample measurement channel, and

a sample collection channel used for collecting a sample that passed through the sample measurement channel,

wherein the first taper channel has a shape that suppresses occurrence of an electroosmotic flow, and

wherein a width of a connection part between the first taper channel and the sample measurement channel is 20 nm to 200 nm.

(2) The device according to (1) described above,

wherein W2/W1 is 10 to 20, where W1 denotes the width of the connection part between the first taper channel and the sample measurement channel, and W2 denotes the width of a connection part between the first taper channel and the sample supply channel, and

wherein L1/W2 is 0.5 to 5, where L1 denotes a length between the connection part between the first taper channel and the sample measurement channel and the connection part between the first taper channel and the sample supply channel.

(3) The device according to (1) or (2) described above, wherein the length of the sample measurement channel is 20 nm to 1000 nm.

(4) The device according to any one of (1) to (3) described above further including a second taper channel arranged between the sample measurement channel and the sample collection channel and having a channel width that increases from the sample measurement channel to the sample collection channel.

(5) The device according to any one of (1) to (4) described above, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

(6) A tunnel current measuring apparatus including: the device according to any one of (1) to (5) described above; an electrophoresis power source; and a measuring unit,

wherein the electrophoresis power source applies a voltage of 10 mV to 5 V to an electrophoresis electrode.

(7) A nucleic acid sequence reading apparatus including: the tunnel current measuring apparatus according to (6) described above; and an analysis unit,

wherein the sample is a nucleic acid, and

wherein the analysis unit identifies a nucleic acid sequence from a measurement result of tunnel current acquired by the tunnel current measuring apparatus.

(8) A tunnel current measuring method using a device,

wherein a device includes

a base material,

a channel formed in the base material, and

a pair of measuring electrodes for measuring tunnel current occurring when a sample passes between the pair of measuring electrodes,

wherein the channel includes

a sample supply channel,

a sample measurement channel in which the measuring electrodes are arranged,

a first taper channel arranged between the sample supply channel and the sample measurement channel and having a channel width that decreases from the sample supply channel to the sample measurement channel, and

a sample collection channel used for collecting a sample that passed through the sample measurement channel,

wherein the first taper channel has a shape that suppresses occurrence of an electroosmotic flow, and

wherein a width of a connection part between the first taper channel and the sample measurement channel is 20 nm to 200 nm,

the tunnel current measuring method including:

a sample electrophoresis step of causing electrophoresis of a sample in the sample supply channel toward the sample collection channel by applying a voltage to the sample supply channel and the sample collection channel; and

a measuring step of measuring tunnel current occurring when a sample passes through a gap between the pair of measuring electrodes arranged in the sample measurement channel.

(9) The tunnel current measuring method according to (8) described above, wherein in the sample electrophoresis step, a voltage of 10 mV to 5 V is applied.

(10) The tunnel current measuring method according to (8) or (9) described above,

wherein W2/W1 is 10 to 20, where W1 denotes the width of the connection part between the first taper channel and the sample measurement channel, and W2 denotes the width of a connection part between the first taper channel and the sample supply channel, and

wherein L1/W2 is 0.5 to 5, where L1 denotes a length between the connection part between the first taper channel and the sample measurement channel and the connection part between the first taper channel and the sample supply channel.

(11) A nucleic acid sequence reading method, wherein the sample is a nucleic acid,

the method including a nucleic acid sequence reading step of identifying a nucleic acid sequence from a measurement result of tunnel current acquired by the measuring step of the tunnel current measuring method according to any one of (8) to (10) described above.

The use of the device disclosed in the present application facilitates a sample to enter the sample measurement channel in which the measuring electrodes are arranged.

### BRIEF DESCRIPTION OF THE DRAWINGS

FIG. 1A is a top view of a device 1.

FIG. 1B is a sectional view on arrow X-X of FIG. 1A.

FIG. 1C is a sectional view on arrow Y-Y of FIG. 1A.

FIG. 2 is a schematic diagram illustrating an example of a fabrication procedure for the device 1.

FIG. 3 is a schematic diagram illustrating an overview of an embodiment of a tunnel current measuring apparatus 100.

FIG. 4 is a schematic diagram illustrating an overview of an embodiment of a nucleic acid sequence reading apparatus 100a.

FIG. 5 is a flowchart of a tunnel current measuring method and a nucleic acid sequence reading method.

FIG. 6A and FIG. 6B are photographs substitute for drawings. FIG. 6A is a SEM photograph near measuring electrodes 4 and a sample measurement channel 32 in which the measuring electrodes 4 are arranged of the device 1 fabricated in Example 1. FIG. 6B is a photograph in which a cover member 5 is bonded to the device 1 and an electrophoresis electrode is inserted therein.

FIG. 7 is a chart representing a measurement result of tunnel current of a nucleic acid measured in Example 4.

FIG. 8A and FIG. 8B are charts representing a measurement result of tunnel current of a nucleic acid measured in Example 5 and Comparative example 1.

FIG. 9 is a graph illustrating a measurement result of a moving velocity of a nucleic acid of Example 6.

FIG. 10 is a graph illustrating a measurement result of a moving velocity of a sample measured in Example 7.

FIG. 11 represents graphs of analyzing the measurement result of FIG. 10.

### DETAILED DESCRIPTION OF SPECIFIC EMBODIMENTS

A device, a tunnel current measuring apparatus, a nucleic acid sequence reading apparatus, a tunnel current measuring method, and a nucleic acid sequence reading method will be described below in detail with reference to the drawings.

In this specification, members having the same type of function are labeled with the same or similar reference symbols. Further, repeated description for the members labeled with the same or similar reference symbols may be omitted.

In this specification, a numerical range expressed by using “to” means a range including numerical values preceding and subsequent to “to” as the lower limit and the upper limit, respectively. A numerical value, a numerical range, and a qualitative expression (for example, an expression of “the same”, “substantially”, or the like) is to be construed as indicating a numerical value, a numerical range, and a nature including an error generally tolerated in the field of the art.

Further, the position, the size, the range, or the like of each component illustrated in the drawings may not necessarily represent an actual position, an actual size, an actual range, or the like for easier understanding. Thus, the disclosure of the present application is not necessarily limited to the position, the size, the range, or the like disclosed in the drawings.

### First Embodiment of Device

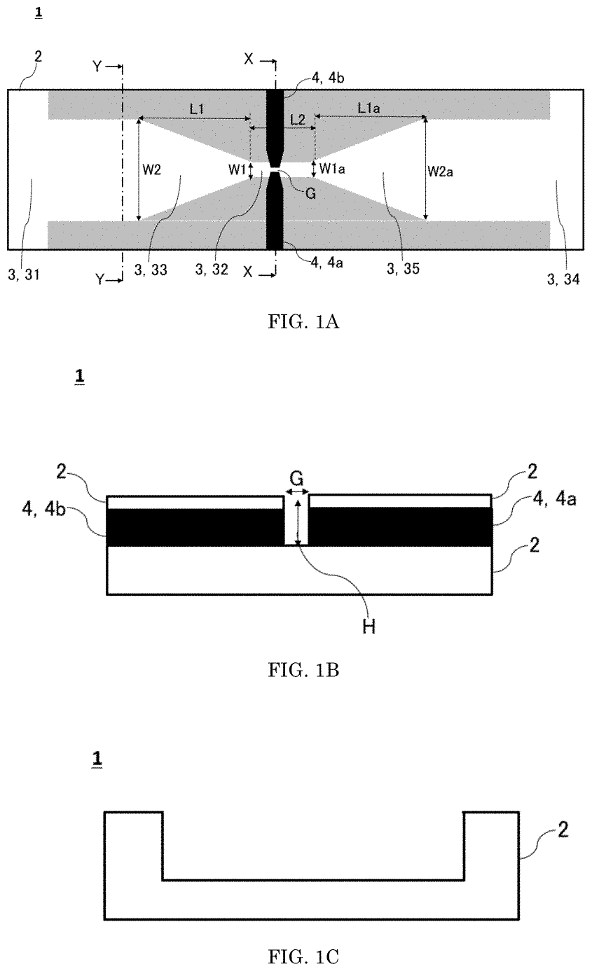

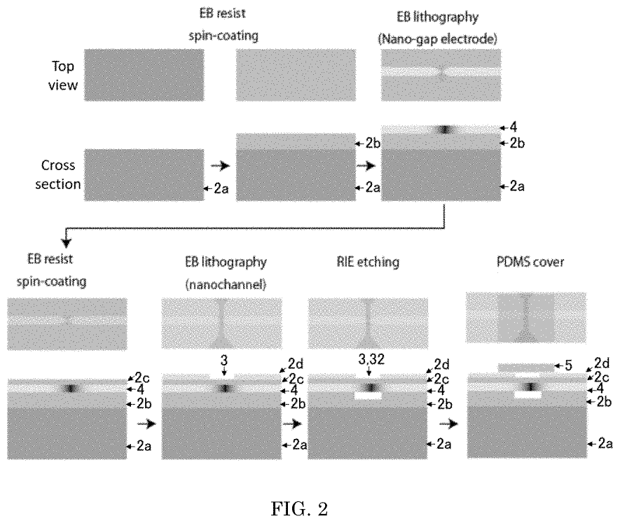

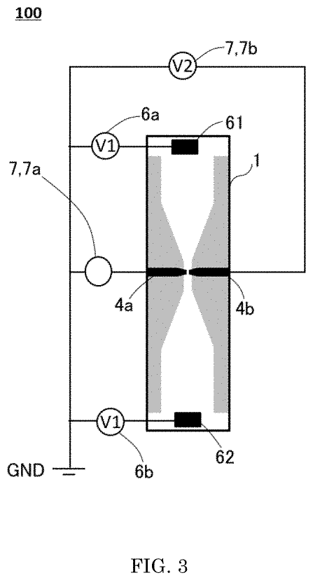

A device 1 according to a first embodiment will be described with reference to FIG. 1A to FIG. 1C. FIG. 1A is a top view of the device 1, FIG. 1B is a sectional view on arrow X-X of FIG. 1A, and FIG. 1C is a sectional view on arrow Y-Y of FIG. 1A. FIG. 2 is a schematic diagram illustrating an example of a fabrication procedure for the device 1.

The device 1 includes a base material 2, a channel 3 formed in the base material 2, and a pair of measuring electrodes 4a and 4b used for measuring tunnel current occurring when a sample passes therebetween (hereafter, the pair of measuring electrodes 4a and 4b may be referred to as “measuring electrode(s) 4”). The channel 3 includes a sample supply channel 31, a sample measurement channel 32 in which the measuring electrodes 4 are arranged, a first taper channel 33 arranged between the sample supply channel 31 and the sample measurement channel 32 and having a channel width decreasing from the sample supply channel 31 to the sample measurement channel 32, and a sample collection channel 34 used for collecting a sample that has passed through the sample measurement channel 32. The width W1 of a connection part between the first taper channel 33 and the sample measurement channel 32 is 20 nm to 200 nm.

Although a second taper channel 35 is depicted in the example illustrated in FIG. 1A, the second taper channel 35 is an optional, additional feature in the device 1 according to the first embodiment. The sample collection channel 34 may be directly coupled to the sample measurement channel 32 as long as a sample flowing out of the sample measurement channel 32 can be collected.

The device 1 can be manufactured by using nanochannel-integrated mechanically controllable break junction, for example. An example of a manufacturing procedure for the device 1 will be illustrated with reference to FIG. 2. Note that mechanically controllable break junction (MCBJ) to fabricate the pair of measuring electrodes 4 is described in Japanese Patent Application Laid-Open No. 2019-525766, Japanese Patent Application Laid-Open No. 2017-509899 described above, M. Tsutsui, K., Shoji, M. Taniguchi, T. Kawai, Nano Lett., 345(2008), M. Tsutsui, M. Taniguchi, T. Kawai, Appl. Phys. Lett. 93, 163115(2008), and the like, for example.

(1) An insulating layer 2b made of an insulating material such as polyimide is formed on a substrate 2a made of silicon or the like.

(2) A metal layer used for forming the measuring electrode 4 is deposited on the insulating layer 2b by electron beam lithography (EB lithography).

(3) A deposition layer 2c made of SiO₂ or the like is formed by chemical deposition. A resist layer 2d is laminated on the deposition layer 2c by spin-coating.

(4) A pattern of the channel 3 including the sample measurement channel 32 is formed by electron beam lithography so as to be overlapped with the metal layer used for forming the measuring electrode 4.

(5) The channel 3 is formed by dry etching. The measuring electrode 4 is then formed by forming a gap (nanogap G) in the metal layer by MCBJ. Note that, although the sample measurement channel 32 is etched up to a part under the measuring electrode 4 in the example illustrated in FIG. 2, there may be no channel in a portion under the measuring electrode 4. Further, although one measuring electrode 4 is provided in the example illustrated in FIG. 2, two or more measuring electrodes 4 may be formed.

(6) A cover member 5 is attached, and one or more holes used for supply of a sample liquid, insertion of an electrophoresis electrode, or the like are formed in the cover member 5, if necessary. Note that the cover member 5 can be attached at least when ion current is measured.

The substrate 2a is not particularly limited as long as it is a material generally used in the field of semiconductor manufacturing technologies. The material of the substrate 2a may be, for example, Si, SiOₓ, SiNₓ, Ge, Se, Te, GaAs, GaP, GaN, InSb, InP, or the like.

The insulating layer 2b is also not particularly limited as long as it is a material generally used in the field of semiconductor manufacturing technologies. The material of the insulating layer 2b may be, for example, an insulating polymer such as polyimide, polypropylene, polyvinyl chloride, polystyrene, high density polyethylene (HDPE), polyacetal (POM), polyepoxy, or the like; an insulating semiconductor metal oxide such as SiO₂, aluminum oxide, or the like; or the like.

The material forming the deposition layer 2c may be an insulating polymer such as polyimide, polypropylene, polyvinyl chloride, polystyrene, high density polyethylene (HDPE), polyacetal (POM), polyepoxy, or the like; an insulating semiconductor metal oxide such as SiO₂, aluminum oxide, or the like; or the like.

The material forming the measuring electrode 4 is not particularly limited as long as it can be used for measuring tunnel current. The material may be, for example, gold, platinum, silver, palladium, tungsten, an alloy of these metals, or the like.

A photoresist used in electron beam lithography and a reagent used in development, etching, and the like are not particularly limited as long as they are materials generally used in the field of micromachining technologies. Further, a spin coater and an apparatus used for etching are also not particularly limited as long as they are devices generally used in the field of micromachining technologies.

The cover member 5 is not particularly limited as long as it is made of a material that can be attached to the base material 2 in which the channel 3 is formed. The material of the cover member 5 may be, for example, polymethyl disiloxane (PDMS) or the like. The cover member 5 and the base material 2 can be attached to each other by ozone plasma treatment or the like, for example.

Note that, in this specification, the term “base material” means a material part that serves as a base used for forming the channel 3. In the example illustrated in FIG. 2, the base material 2 includes the substrate 2a, the insulating layer 2b, the deposition layer 2c, and the resist layer 2d. Note that FIG. 2 merely illustrates one example of the fabrication procedure for the device 1 in which the measuring electrodes 4 are arranged in the sample measurement channel 32. There may be addition of another step or deletion of some of the above steps as long as the device 1 achieves the advantageous effect disclosed in the present application. For example, the resist layer 2d may be removed after the channel 3 is formed by etching. In such a case, the resist layer 2d is not included in the base material 2. Further, the device 1 may be fabricated by an electron beam engraving method, nano-printing, or the like.

Further, for easier understanding, detailed depiction of the substrate 2a, the insulating layer 2b, the deposition layer 2c, and the resist layer 2d is omitted in the example illustrated in FIG. 1A to FIG. 1C, and these components are depicted as the base material 2.

Application of a voltage to the sample supply channel 31 and the sample collection channel 34 causes electrophoresis force to be provided to a sample and increases the moving velocity of the sample. This results in an improved measuring rate of a sample compared to a case where no electrophoresis force is provided. In contrast, when a voltage is applied to the channel 3 to provide electrophoresis force to a sample, a larger sectional area of the channel 3 requires a larger voltage.

Reading of a nucleic acid from tunnel current is performed by identifying a difference in the measured current value in the order of picoampere. The present inventors have newly found from various experiment results that, when a voltage at a level that can provide electrophoresis force is applied to a nucleic acid supplied in the channel of the order of micrometer disclosed in Application Laid-Open No. 2016-103979, the measuring electrode 4 detects noise due to the voltage applied for electrophoresis and is unable to identify a nucleic acid. The disclosure of the present application is based on this finding.

The device 1 disclosed in the present application can provide electrophoresis force to a sample at a low voltage and thus can measure tunnel current with less noise due to the voltage applied for electrophoresis. Therefore, the device 1 can be preferably used for identifying a nucleic acid as described above, and the sample is not limited to a nucleic acid. With any sample having surface charges and moved by electrophoresis, tunnel current with less noise due to a voltage applied for electrophoresis can be measured. The sample may be, for example, a peptide, a lipid, a glycan, a synthetic polymer, or the like. Note that, in this specification, when “sample liquid” is referred to, the “sample liquid” means a liquid in which the sample described above is dissolved or dispersed in a solvent used for electrophoresis.

The device 1 requires the sample supply channel 31 having a predetermined size for supplying a sample liquid thereto. Thus, the device 1 employs the structure in which the width of the sample measurement channel 32 in which the measuring electrodes 4 are arranged is made narrower (smaller) and the sample supply channel 31 and the sample measurement channel 32 are connected via the first taper channel 33.

As described above, to reduce noise due to a voltage applied for electrophoresis, it is preferable that the width of the sample measurement channel 32 be narrower. When the width of the connection part between the first taper channel 33 and the sample measurement channel 32 is denoted as W1, W1 can be 200 nm or less, 180 nm or less, 160 nm or less, 140 nm or less, 120 nm or less, 100 nm or less, 90 nm or less, 80 nm or less, 70 nm or less, 60 nm or less, or 50 nm or less. On the other hand, there is no limitation in the lower limit of W1 as long as it is within a manufacturable range, and the lower limit of W1 can be, but is not limited to, 20 nm or greater, 25 nm or greater, or 30 nm or greater, for example.

The width of the sample measurement channel 32 may be the same along the entire length or may vary along the length as long as it is within a range that does not affect analysis of a measurement result or the like. In the example illustrated in FIG. 1A, when the end opposite to the width W1 of the sample measurement channel 32 is denoted as W1a, W1a may be the same as W1 or may be larger or smaller than W1.

The gap between the pair of measuring electrodes 4a and 4b (gap G, see FIG. 1B) is not particularly limited as long as it is within the range that enables measurement of tunnel current occurring when a sample passes therebetween. The gap G can be, but is not limited to, 0.1 nm or greater, 0.3 nm or greater, 0.5 nm or greater, 0.7 nm or greater, or 0.9 nm or greater, for example. On the other hand, the upper limit of the gap G can be, but is not limited to, 50 nm or less, 30 nm or less, 10 nm or less, 8 nm or less, 6 nm or less, 5 nm or less, 4 nm or less, 3 nm or less, 2 nm or less, or 1 nm or less, for example.

The length of the measuring electrode 4 (the length of the gap G in the same direction as L2 of FIG. 1A) is also not particularly limited as long as it is within a range that enables measurement of tunnel current occurring when a sample passes therebetween. The length can be, but is not limited to, 1000 nm or less, 800 nm or less, 600 nm or less, 400 nm or less, 200 nm or less, 100 nm or less, 80 nm or less, or 60 nm or less, for example.

Note that, for easier cutting in MCBJ, a smaller deposition amount of the measuring electrodes 4 (in a direction orthogonal to the direction of the length of the measuring electrode 4 or in the direction H in FIG. 1B, hereafter, which may be denoted as “thickness”) is preferable. An increase in the thickness of the measuring electrode 4 may make it difficult to control a cutting place and result in a rough cut surface of the fabricated gap G. Thus, the thickness of the measuring electrode 4 can be, but is not limited to, 80 nm or less, 70 nm or less, 60 nm or less, or 50 nm or less, for example. The lower limit of the thickness of the measuring electrode 4 is not particularly limited as long as tunnel current can be measured and can be, but is not limited to, 2 nm or greater, 4 nm or greater, 6 nm or greater, 8 nm or greater, 10 nm or greater, 15 nm or greater, or 20 nm or greater, for example.

As described above, it is preferable that the length of the measuring electrode 4 be larger than the thickness thereof in order to reduce the thickness of the measuring electrode 4 to form the gap G by MCBJ. The ratio of length/thickness may be, but is not limited to, 10 to 100, for example.

Electrodes are formed by MCBJ also for the device disclosed in Japanese Patent Application Laid-Open No. 2017-509899. Thus, the thickness of the electrode deposited on the thin film is thin for the reason described above. Further, in the device disclosed in Japanese Patent Application Laid-Open No. 2017-509899, since nanopores are formed in the thin film, a sample moves in the thickness direction of the gap G of the electrodes. On the other hand, when the device 1 disclosed in the present application is used to measure tunnel current of a sample, the sample moves in the longitudinal direction of the measuring electrodes 4. That is, the moving direction of a sample with respect to the gap G differs between the device 1 disclosed in the present application and the device disclosed in Japanese Patent Application Laid-Open No. 2017-509899. In the case of the device disclosed in the present application, since an electric field is applied in the longitudinal direction of the measuring electrodes 4, this results in a gradual intensity of the electric field and easier control of the moving velocity of a sample. In contrast, in the case of the device of Japanese Patent Application Laid-Open No. 2017-509899, since an electric field is applied in the thickness direction of the electrode (the thickness of the electrode is smaller than the length of the electrode), this results in a steep electric field and makes it difficult to control the moving velocity of a sample. As set forth, the device 1 disclosed in the present application achieves an advantageous effect of easier control of the moving velocity of a sample passing through the gap G of the measuring electrodes 4 compared to the device disclosed in Japanese Patent Application Laid-Open No. 2017-509899.

The length L2 of the sample measurement channel 32 is not particularly limited as long as it is within a range that enables measurement of tunnel current occurring when a sample passes therethrough. If the length L2 is too long, the entire channel of the device 1 will be longer. In contrast, if the length L2 is too short, when the sample is an elongate sample (hereafter, also referred to as “elongate sample”) such as a nucleic acid or a peptide, it will be difficult to maintain a state where an elongate sample is elongated. The length L2 can be, but is not limited to, 20 nm or greater, 25 nm or greater, 30 nm or greater, 35 nm or greater, 40 nm or greater, 45 nm or greater, or 50 nm or greater. Further, the length L2 can be 2000 nm or less, 1500 nm or less, 1000 nm or less, 800 nm or less, 600 nm or less, 400 nm or less, 200 nm or less, 180 nm or less, 160 nm or less, 140 nm or less, 120 nm or less or 100 nm or less. The length L2 is naturally required to be longer than the length of the gap G part of the measuring electrodes 4.

To reduce noise due to a voltage applied for electrophoresis, it is preferable that the depth H of the channel 3 be also smaller. The depth H of the channel 3 can be, but is not limited to, 200 nm or less, 180 nm or less, 160 nm or less, 140 nm or less, 120 nm or less, 100 nm or less, 90 nm or less, 80 nm or less, 70 nm or less, 60 nm or less, or 50 nm or less, for example. On the other hand, the depth H of the channel 3 can be 20 nm or greater, 25 nm or greater, or 30 nm or greater, for example.

In the device 1 according to the first embodiment, while the length of the first taper channel 33 (L1 in FIG. 1A) and the width of the sample supply channel 31 (the connection part to the first taper channel 33, W2 in FIG. 1A) are not particularly limited, the width of the channel 3 is desirably as small as possible. Note that the sample supply channel 31 may have a wider part having a width larger than W2, if necessary, for supplying a sample liquid.

In the device 1 according to the first embodiment, while the width of the sample collection channel 34 and the length of the optionally, additionally provided second taper channel 35 (L1a in FIG. 1A) are not particularly limited, the width of the channel 3 is desirably as small as possible. Note that, to collect a sample, the sample collection channel 34 may have a wider part having a width larger than W2a, if necessary.

In comparison to the devices disclosed in Japanese Patent Application Laid-Open No. 2017-509899 and Japanese Patent Application Laid-Open No. 2016-103979, the following advantageous effects are achieved when the device 1 according to the first embodiment is used to measure tunnel current.

(1) A sample contained in a sample liquid supplied to the sample supply channel 31 is guided to the sample measurement channel 32 via the first taper channel 33 by electrophoresis. Therefore, even with a small amount of a sample such as a nucleic acid contained in the sample liquid, the sample can be more reliably guided to the measuring electrodes 4 than in the nanopore scheme disclosed in Japanese Patent Application Laid-Open No. 2017-509899.

(2) The nanopore of the device disclosed in Japanese Patent Application Laid-Open No. 2017-509899 is a three-dimensional hole formed in a thin film. It is thus very difficult to change the size of the hole inside the nanopore. Further, in the device disclosed in Japanese Patent Application Laid-Open No. 2017-509899, a chamber is formed such that a sample liquid comes into contact with the thin film. It is thus very difficult to design openings of the chamber and the nanopore without a level difference therebetween. That is, there is a limitation in channel design to facilitate a sample such as a nucleic acid contained in a sample liquid to flow into the openings. In contrast, in the device 1, since the channel 3 is formed in the base material 2, the channel 3 having a desired shape can be easily formed by electron beam lithography.

(3) The value of the width of the sample measurement channel 32 is made significantly small and a taper is formed from the sample supply channel 31 to the sample measurement channel 32, and thereby the sectional area of the channel 3 can be smaller than in the embodiment disclosed in Japanese Patent Application Laid-Open No. 2016-103979. Thus, a voltage for providing electrophoresis force to a sample can be reduced, and noise caused by the voltage applied for electrophoresis can be reduced when tunnel current is measured.

### Optional, Additional Modified Example of Device 1

An optional, additional modified example (limitation) of the device 1 will be described with reference to FIG. 1A to FIG. 1C. Note that the optional, additional modified example of the device 1 is an embodiment that further limits each feature of the embodiment of the device 1. Thus, only the limitations will be described for the optional, additional modified example of the device 1, and repeated description for the features already described in the first embodiment will be omitted.

### Modified Example 1

In the device 1, when the width of the connection part between the first taper channel 33 and the sample measurement channel 32 is denoted as W1, and the width of the connection part between the first taper channel 33 and the sample supply channel 31 is denoted as W2, the lower limit of W2/W1 may be 2 or greater, 3 or greater, 4 or greater, 5 or greater, 6 or greater, 7 or greater, 8 or greater, 9 or greater, or 10 or greater, and the upper limit of W2/W1 may be 50 or less, 40 or less, 30 or less, or 20 or less. Further, when the length between the connection part between the first taper channel 33 and the sample measurement channel 32 and the connection part between the first taper channel and the sample supply channel is denoted as L1, the lower limit of L1/W2 may be 0.3 or greater, 0.4 or greater, or 0.5 or greater, and the upper limit of L1/W2 may be 10 or less, 9 or less, 8 or less, 7 or less, 6 or less, or 5 or less.

When the shape of the first taper channel 33 is in accordance with the ratio described above, the following advantageous effects are achieved in addition to the advantageous effects described for the device 1 according to the first embodiment.

(1) With the first taper channel 33 being formed at the ratio described above, this can facilitate an elongate sample in a sample liquid to be straightened.

(2) While an electroosmotic flow (EOF) occurs inside the channel when the channel is filled with a solvent and a voltage is applied thereto, a reverse flow occurs in a region along the wall. When the first taper channel 33 is formed in the range described in the modified example 1, occurrence of the EOF is likely to be suppressed, and an acceleration effect due to an enhanced electric field is obtained because of the shape of the first taper channel 33. Therefore, the elongate sample contained in a sample liquid is straightened and is likely to be introduced in the sample measurement channel 32.

### Modified Example 2

The device 1 may include the second taper channel 35 arranged between the sample measurement channel 32 and the sample collection channel 34 and having the channel width increasing from the sample measurement channel 32 to the sample collection channel 34.

When the device 1 includes the second taper channel 35, an advantageous effect of preventing an elongate sample from being stacked at the outlet of the sample measurement channel 32 to facilitate passage of the elongate sample is provided in addition to the advantageous effects described for the device 1 according to the first embodiment and modified example 1.

### Modified Example 3

In addition to the limitation to modified example 2, the device 1 may satisfy W1=W1a, W2=W1, and L1=L1a, where W1a denotes the width of the connection part between the sample measurement channel 32 and the second taper channel 35, W2a denotes the width of the connection part between the second taper channel 35 and the sample collection channel 34, and L1a denotes the length between the connection part between the second taper channel 35 and the sample measurement channel 32 and the connection part between the second taper channel 35 and the sample collection channel 34. In other words, the channel 3 may be formed to be symmetrical about the sample measurement channel 32.

When the channel 3 of the device 1 is formed to be symmetrical about the sample measurement channel 32, the following advantageous effects are achieved in addition to the advantageous effects described for the device 1 according to the first embodiment, modified example 1, and modified example 2.

(1) By exchanging the positive pole and the negative pole of the electrophoresis electrodes, it is also possible to measure a sample passing between the measuring electrodes 4 from the reverse direction. For example, since it is possible to confirm the same sequence from different directions when reading the sequence of a nucleic acid, a peptide, or the like, improvement in reading accuracy is expected.

### Other Modified Examples

The device 1 disclosed in the present application is not limited to the first embodiment and modified examples 1 to 3 described above and may be modified or changed as appropriate within the scope of the technical concept disclosed in the present application. Also, some of the components can be omitted in each embodiment.

For example, the manufactured device 1 may be hydrophilized so as to facilitate flow of a sample liquid. The hydrophilizing method may be plasma treatment, surfactant treatment, polyvinyl pyrrolidone (PVP) treatment, photocatalytic treatment, SiO₂ film coating, or the like. For example, it is possible to introduce a hydroxy group to the surface by performing plasma treatment for 10 to 30 seconds on the surface of the device 1 on which the channel 3 is formed. Further, the device 1 may have the cover member 5. Furthermore, electrodes for applying voltages for electrophoresis may be formed to the sample supply channel 31 and the sample collection channel 34 of the device 1. The electrophoresis electrode will be described later.

### Embodiment of Tunnel Current Measuring Apparatus

An embodiment of the tunnel current measuring apparatus 100 will be described with reference to FIG. 3. FIG. 3 is a schematic diagram illustrating an overview of the embodiment of the tunnel current measuring apparatus 100. The tunnel current measuring apparatus 100 includes an electrophoresis power source (hereafter, also referred to as “first power source”) 6 and a measuring unit 7 in addition to the device 1. The measuring unit 7 includes a tunnel current detection unit (hereafter, also referred to as “detection unit”) 7a and a tunnel current measuring power source (hereafter, also referred to as “second power source”) 7b.

In the example illustrated in FIG. 3, an electrophoresis first electrode (hereafter, also referred to as “first electrode”) 61 is formed at a part in contact with a sample liquid inside the sample supply channel 31, and an electrophoresis second electrode (hereafter, also referred to as “second electrode”) 62 is formed at a part in contact with a solvent inside the sample collection channel 34. The first electrode 61 and the second electrode 62 may be components of the device 1 or may be components of the tunnel current measuring apparatus 100.

The first electrode 61 and the second electrode 62 can be formed of a known conductive metal such as Ag/AgCl, aluminum, copper, platinum, gold, silver, titanium, or the like. The first electrode 61 and the second electrode 62 can may be formed on the base material 2 or may be a separate member from the device 1 and inserted via a hole of the cover member 5.

FIG. 3 illustrates an example in which two first power sources 6 of a first power source 6a connected to the first electrode 61 and a first power source 6b connected to the second electrode 62 are used to apply voltages for electrophoresis. In the example illustrated in FIG. 3, since two power sources are used as the first power source 6, the voltages can be separately increased and decreased. Note that the example illustrated in FIG. 3 is a mere example, and the disclosure is not limited thereto. A single first power source 6 may be provided, for example, as long as a voltage for electrophoresis described later can be applied. In the tunnel current measuring apparatus 100 disclosed in the present application, with a significantly smaller width of the channel 3, in particular, the sample measurement channel 32 of the device 1, a voltage required for electrophoresis of a sample can be reduced. Thus, the measuring unit 7 can obtain a measurement value of tunnel current with a small noise component when measuring tunnel current occurring when a sample passes through the gap between the measuring electrodes 4. Therefore, the obtained measurement result can be preferably used for use of identification of the sequence of a nucleic acid or a peptide, analysis of a lipid, a glycan, or a synthetic polymer, or the like.

If the voltage applied by the first power source 6 is too low, the moving velocity of a sample is slow, and a long time is required for measurement. The voltage applied by the first power source 6 can be, but is not limited to, 10 mV or higher, 15 mV or higher, 20 mV or higher, 25 mV or higher, or 30 mV or higher, for example. On the other hand, the upper limit of the voltage applied by the first power source 6 can be set as appropriate taking into consideration of accuracy of an analysis unit when an obtained measurement result is analyzed, the width of the channel 3, and the like. The upper limit of the voltage applied by the first power source 6 can be, but is not limited to, 5 V or lower, 3 V or lower, 1 V or lower, 500 mV or lower, 300 mV or lower, 100 mV or lower, 90 mV or lower, 80 mV or lower, 70 mV or lower, 60 mV or lower, or 50 mV or lower, for example. Note that a measurement result of tunnel current obtained by the tunnel current measuring apparatus 100 may be analyzed by an analysis unit that is a separate component from the tunnel current measuring apparatus 100. Therefore, the analysis unit is not an essential component in the tunnel current measuring apparatus 100.

A detection unit 7a of the measuring unit 7 is not particularly limited as long as it has a component that can measure a change in tunnel current occurring when a sample passes through the gap between the pair of measuring electrodes 4a and 4b across which a voltage is applied by a tunnel current measuring power source (hereafter, also referred to as “second power source”) 7b. For example, since a change in occurring tunnel current is of the order of picoampere, a known ammeter that can measure current of the order of picoampere can be used. Further, the current may be calculated from a voltage measured by a voltmeter. The measuring unit 7 may optionally, additionally include a current amplifier, a noise removal device, an analog-to-digital (A/D) converter, or the like. When the measuring unit 7 includes a current amplifier, a noise removal device, an A/D converter, or the like, data that will be easily analyzed can be provided instead of raw data of measured tunnel current values. Alternatively, the measuring unit 7 may have only the component that can measure a change in tunnel current, and the current amplifier, the noise removal device, the A/D converter, or the like may be components of the analysis unit 8.

The second power source 7b applies a voltage across the pair of measuring electrodes 4a and 4b. The voltage applied by the second power source 7b is not particularly limited as long as tunnel current can be measured. The lower limit of the voltage applied by the second power source 7b can be, but is not limited to, 20 mV or higher, 50 mV or higher, or 100 mV or higher, and the upper limit thereof can be, but is not limited to, 750 mV or lower, 500 mV or lower, 250 mV or lower, or the like, for example. A specific configuration of the second power source 7b is not particularly limited, and a known power source device can be used.

The use of the tunnel current measuring apparatus 100 disclosed in the present application to measure tunnel current achieves an advantageous effect that a measurement result of tunnel current with a less noise component due to a voltage applied for electrophoresis can be obtained.

### Embodiment of Nucleic Acid Sequence Reading Apparatus

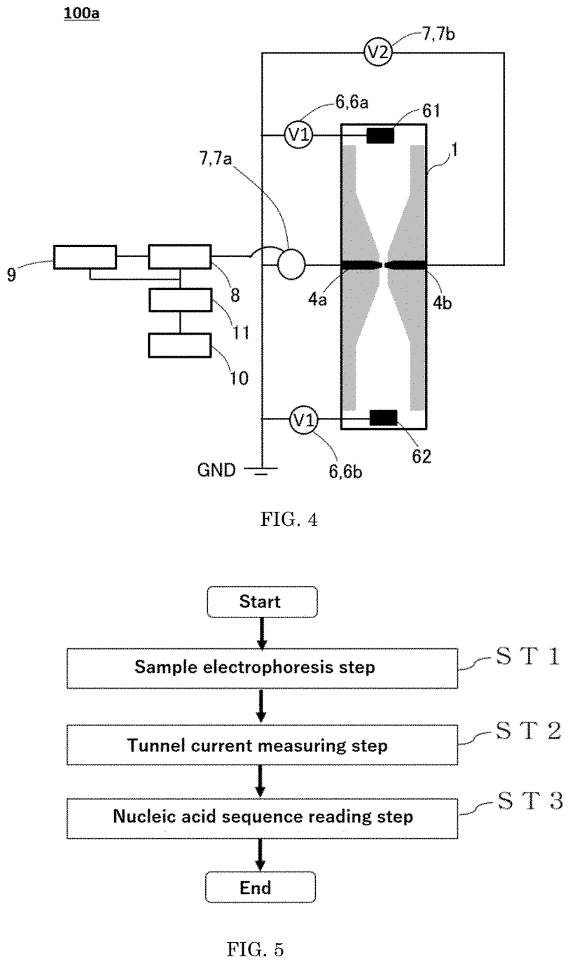

An embodiment of a nucleic acid sequence reading apparatus 100a will be described with reference to FIG. 4. FIG. 4 is a schematic diagram illustrating an overview of the embodiment of the nucleic acid sequence reading apparatus 100a. The nucleic acid sequence reading apparatus 100a is the same as the tunnel current measuring apparatus 100 except that the nucleic acid sequence reading apparatus 100a includes, in addition to the tunnel current measuring apparatus 100, the analysis unit 8 as an essential component that identifies a nucleic acid sequence from a measurement result of tunnel current acquired by the tunnel current measuring apparatus 100. Thus, the analysis unit 8 will be mainly described in the embodiment of the nucleic acid sequence reading apparatus 100a, and repeated description for the features already described in the embodiment of the tunnel current measuring apparatus 100 will be omitted. Thus, even without explicit description in the embodiment of the nucleic acid sequence reading apparatus 100a, naturally, the features already described in the embodiment of the tunnel current measuring apparatus 100 can be employed.

The analysis unit 8 identifies a nucleic acid sequence from a measurement result of tunnel current acquired by the tunnel current measuring apparatus 100. More specifically, the analysis unit 8 calculates conductance from a measurement value of tunnel current. When tunnel current is measured, the conductance can be calculated by dividing a measurement value of the tunnel current by a voltage applied across the pair of measuring electrodes 4a and 4b. The conductance calculated from tunnel current occurring when a nucleic acid passes between the pair of measuring electrodes 4a and 4b varies in accordance with the type of the nucleic acid. Therefore, since the type of a nucleic acid can be identified based on calculated conductance, a nucleic acid sequence can be read through time series analysis of measurement values of tunnel current.

Note that the analysis unit 8 may perform up to the conductance analysis described above, and a separate device from the analysis unit 8 may perform identification of a specific nucleic acid name, such as adenine (A), guanine (G), cytosine (C), thymine (T), uracil (U), or the like. Alternatively, the analysis unit 8 may be provided with a storage unit that stores conductance corresponding to types of nucleic acids, and the analysis unit 8 may directly read a nucleic acid sequence from a measurement result of tunnel current by comparing calculated conductance with conductance written in the storage unit. In this specification, “identifying a nucleic acid sequence” encompasses providing conductance information that can identify a type of a nucleic acid in addition to specifically identifying a type of a nucleic acid.

Note that, in this specification, “nucleic acid” includes an artificial nucleic acid in addition to the nucleic acids described above forming DNA and RNA. Examples of the artificial nucleic acid may be, but is not limited to, the following artificial nucleic acids, for example.

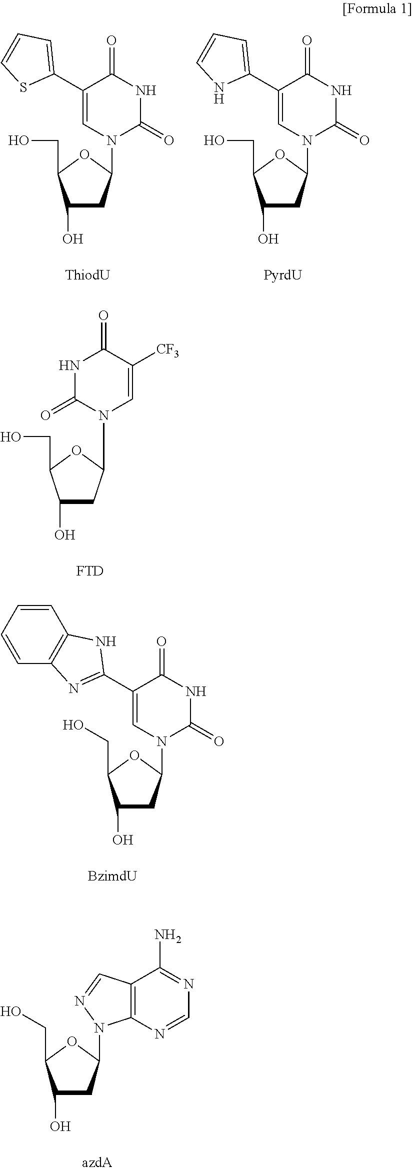

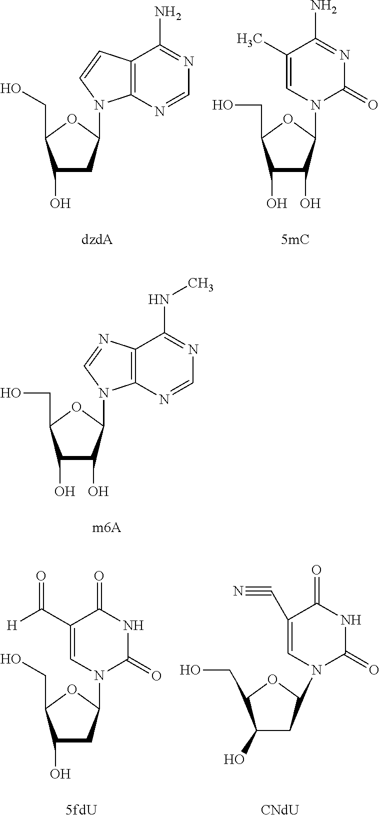

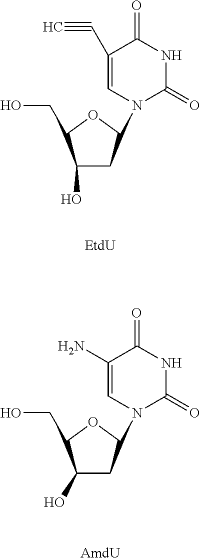

Addition of different types of artificial nucleic acids to natural nucleic acids can increase the density of a nucleic acid memory by increased bits.

The nucleic acid sequence reading apparatus 100a may optionally, additionally include a display unit 9 that displays a measured tunnel current value and/or a result analyzed by the analysis unit 8, a program memory 10 that stores a program in advance that causes the analysis unit 8 or the display unit 9 to function, and a control unit 11 that reads and executes the program stored in the program memory 10. The program may be stored in the program memory 10 in advance or may be stored in a storage medium and then stored in the program memory 10 by using installing means.

As the display unit 9, a known display device such as a liquid crystal display, a plasma display, an organic EL display, or the like can be used.

### Embodiment of Tunnel Current Measuring Method and Nucleic Acid Sequence Reading Method

Next, a tunnel current measuring method using the tunnel current measuring apparatus 100 and a nucleic acid sequence reading method using the nucleic acid sequence reading apparatus 100a will be described with reference to FIG. 5. FIG. 5 is a flowchart of the tunnel current measuring method and the nucleic acid sequence reading method. The embodiment of the tunnel current measuring method includes a sample electrophoresis step (ST1) and a tunnel current measuring step (ST2). The embodiment of the nucleic acid sequence reading method includes a nucleic acid sequence reading step (ST3) in addition to the sample electrophoresis step (ST1) and the tunnel current measuring step (ST2).

The sample electrophoresis step (ST1) is performed by supplying a sample liquid to the sample supply channel 31, supplying a solvent to the sample collection channel 34, and applying voltages to the first electrode 61 and the second electrode 62. The supplied sample liquid or the solvent is permeated by capillary force and thereby liquid junction is provided in in the first taper channel 33, the sample measurement channel 32, and the second taper channel 35 formed if necessary. The solvent used for fabricating the sample liquid can be any conductive solvent. The solvent may be, but is not limited to, ultrapure water, a buffer liquid, or the like, for example. The ultrapure water can be manufactured by using Milli-Q (registered trademark) Integral 3 (device name) manufactured by EMD Millipore (Milli-Q (registered trademark) Integral 33/5/1015 (catalog number)), for example. The buffer liquid may be a known buffer for electrophoresis, such as TE buffer, TBE buffer, or the like. The concentration of the buffer can be adjusted as appropriate within a range that enables electrophoresis, such as 1 μM or less, for example, without being limited thereto. Further, the sample liquid may be a surfactant such as polyvinyl-pyrrolidone (PVP) or otherwise an amphiphilic chemical, if necessary, in order to reduce influence of an electroosmotic flow (EOF).

In the tunnel current measuring step (ST2), tunnel current occurring when the sample passes through the gap between the pair of measuring electrodes 4a and 4b arranged in the sample measurement channel 32 is measured.

The nucleic acid sequence reading step (ST3) is performed when the sample is a nucleic acid. In the nucleic acid sequence reading step (ST3), a nucleic acid sequence is identified by the method described above (in the embodiment of the nucleic acid sequence reading apparatus) from the tunnel current value obtained by the tunnel current measuring step (ST2). Note that the electrophoresis step (ST1), the tunnel current measuring step (ST2), and the nucleic acid sequence reading step (ST3) may be performed in advance by using a nucleic acid having a known sequence, and the type of a nucleic acid and the calculated conductance may be associated with each other and stored in the storage unit, if necessary. Further, when the sample is a peptide, “nucleic acid” can be replaced with “amino acid”.

While details of the disclosure of the present application will be specifically described with Examples below, each Example is intended to provide a reference for a specific aspect. These illustrations are intended to neither limit nor express to limit the scope of the disclosure in the present application.

### EXAMPLES

Fabrication of Device 1

### Example 1

The device was fabricated in accordance with the procedure illustrated in FIG. 2. The specific procedure was as follows.

(1) The polyimide insulating layer 2b was formed on the silicon substrate 2a.

(2) A metal layer used for forming the measuring electrode 4 on the insulating layer 2b was deposited on the insulating layer 2b by using electron beam lithography and lift-off technology. ZEP520A was used for the resist, and gold was used for the material of the metal layer used for forming the measuring electrode 4.

(3) The SiO₂ deposition layer 2c was formed by chemical deposition. The resist layer 2d was laminated on the deposition layer 2c by spin-coating. ZEP520A was used for the resist.

(4) The pattern of the channel 3 including the sample measurement channel 32 was formed so as to overlap the metal layer used for forming the measuring electrode 4 by electron beam lithography.

(5) The channel 3 was formed by dry etching. The substrate 2a was folded to form a gap (nanogap G) in the material layer by MCBJ, and thereby the measuring electrode 4 was formed.

(6) The cover member 5 made of PDMS in which a supply hole for a sample liquid and an insertion hole for an electrophoresis electrode were formed was fabricated by electron beam lithography. The base material 2 in which the channel 3 was formed and the cover member 5 were treated by ozone plasma and bonded to each other. Ag/AgCl was used for the electrophoresis electrode, and the electrophoresis electrode was inserted from a hole formed in the cover member 5.

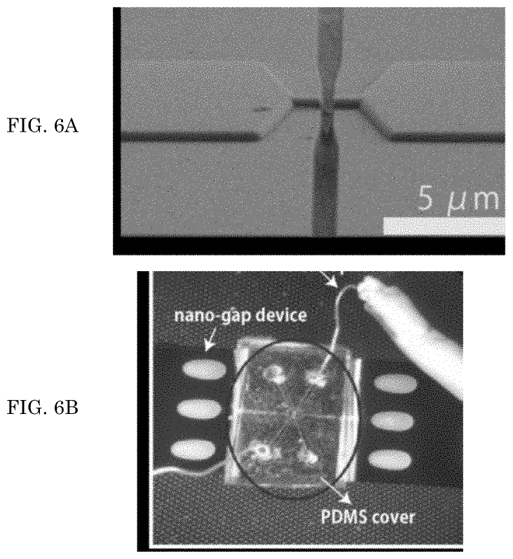

FIG. 6A is a SEM photograph near the measuring electrodes 4 and the sample measurement channel 32 in which the measuring electrodes 4 are arranged of the device 1 fabricated in Example 1. FIG. 6B is a photograph in which the cover member 5 is bonded to the device 1 and an electrophoresis electrode is inserted therein.

The dimensions of the device 1 illustrated in FIG. 6A will be described with reference to the reference symbols of FIG. 1A. The width W1 of the connection part between the first taper channel 33 and the sample measurement channel 32 was 200 nm, the length L2 of the sample measurement channel 32 was 8 μm, the width W2 of the connection part between the first taper channel 33 and the sample supply channel 31 was 2 μm, and the length L1 of the first taper channel 33 was 5 μm, and W1=W1a, W2=W2a, and L1=L1a were satisfied. Further, the gap G between the pair of the measuring electrodes 4a and 4b was adjusted to be 0.55 nm to 1.0 nm. Further, the depth of the channel 3 was 50 nm.

### Example 2

The mask for electron beam lithography was changed to fabricate the device so that the same ratio as that of Example 1 is obtained while the width of the channel is smaller than that of Example 1. The size of the device fabricated in Example 2 satisfied W1=W1a=20 nm, W2=W2a=200 nm, L1=L1a=500 nm, and L2=800 nm. Further, the gap G between the pair of the measuring electrodes 4a and 4b was 1 nm, and the depth of the channel 3 was 20 nm.

### Example 3

Fabrication of Nucleic Acid Sequence Reading Apparatus (Tunnel Current Measuring Apparatus)

A battery was used as the electrophoresis power source 6 and connected via lead wires to the electrophoresis electrodes of the device fabricated in Example 2. In the tunnel current detection unit 7a of the measuring unit 7, a scheme to obtain a current value by performing current/voltage amplification to measure a micro-current value as a voltage was used for the ammeter, and a digital oscilloscope by National Instrument that is an A/D converter was used as a voltmeter. Further, a feedback resistor was incorporated in a commercially available current amplifier to increase accuracy of a current amplifier.

Tunnel Current Measuring Method and Nucleic Acid Sequence Reading Method

### Example 4

(1) Preparation of a Sample Liquid

As a nucleic acid, λDNA (NIPPON GENE CO., LTD., Tokyo, Japan) having a known sequence was used. Water was used as a solvent, and the nucleic acid was dissolved in the solvent to prepare a sample liquid. The concentration of the nucleic acid was 1 micro-mol/l.

(2) Measurement of Tunnel Current (Acquisition of Nucleic Acid Information)

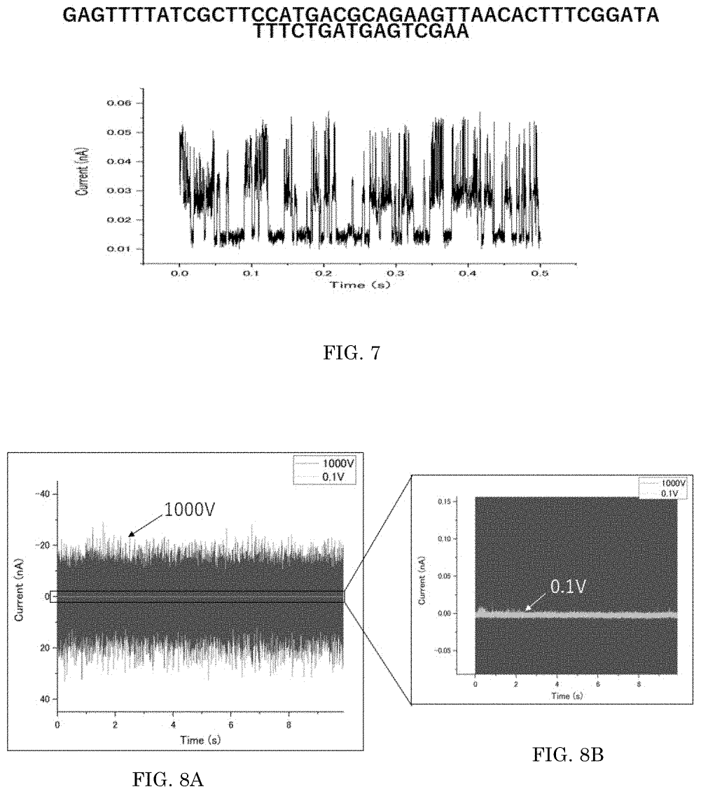

The sample liquid prepared in (1) described above was supplied to the sample supply channel, and the solvent was supplied to the sample collection channel. DC voltages 600 mV and −600 mV were applied to the electrophoresis electrodes 61 and 62, respectively. A DC voltage of 100 mV was applied to the measuring electrodes 4. FIG. 7 indicates a measurement result.

(3) Reading of a Nucleic Acid Sequence

The conductance was calculated from the waveform of the tunnel current of (2) described above. The nucleic acid sequence was determined from the time axis and the calculated conductance and then confirmed to be the same as the sequence of λDNA used as the sample.

### Example 5, Comparative Example 1

The tunnel current was measured in the same procedure as in Example 4 except that the DC voltages applied to the electrophoresis electrodes 61 and 62 were changed to 0.1 V (Example 5) and 1000 V (Comparative example 1). FIG. 8 indicates a measurement result. As illustrated in FIG. 8A, when a voltage for electrophoresis of 1000 V was applied, a difference in tunnel current in the order of picoampere occurring when the nucleic acid passes through the gap G between the measuring electrodes 4 was not identified at all due to noise. FIG. 8B is a graph of an enlarged part near the picoampere order range of the measurement result of the tunnel current value of FIG. 8A. As illustrated in FIG. 8B, when the voltage was 0.1 V, a difference in tunnel current in the order of picoampere occurring when the nucleic acid passes through the gap G between the measuring electrodes 4 was identified.

It was confirmed from the above results that, if the voltage applied for electrophoresis is high, a difference in tunnel current in the order of picoampere occurring when the nucleic acid passes through the gap G between the measuring electrodes 4 is unable to be identified due to noise. As disclosed in Japanese Patent Application Laid-Open No. 2016-103979, when a channel formed in a substrate is used for elongation of a nucleic acid, the width of the channel and the applied voltage can be set to values that enable elongation of the nucleic acid. In general, however, a larger width of the channel requires a larger voltage value for the nucleic acid to move inside the channel by electrophoresis. It is known in the field of micromachining that a micro-channel can be formed in a substrate. However, the device disclosed in the present application achieves a significant advantageous effect that, with improved size and arrangement of the channel, a nucleic acid sequence can be read from tunnel current by using a channel formed in a base material.

Moving Velocity of Nucleic Acid in Accordance with Adjustment of Sample Liquid

### Example 6

Next, an experiment was performed to confirm a flow of a nucleic acid due to electrophoresis when the first taper channel 33 was formed.

(1) Fabrication of a Device

A device having a larger channel than the device fabricated in Example 2 was fabricated in order to observe a stained nucleic acid by using an optical microscope. Note that the measuring electrodes 4 were not formed in Example 6, because the experiment was not intended to read a nucleic acid sequence. The device was fabricated in accordance with the procedure of Example 1 except that the measuring electrodes 4 were not formed. The size of the device fabricated in Example 6 satisfied W1=W1a=1 μm, W2=W2a=10 μm, L1=L1a=20 μm, and L2=20 μm. The depth of the channel 3 was 20 nm.

(2) Adjustment of a Sample Liquid

As a nucleic acid, λDNA (NIPPON GENE CO., LTD., Tokyo, Japan) was used. The purchased λDNA was used as it stands without purification. The λDNA was stained with YOYO-1 (registered trademark) Iodide (Thermo Fisher Scientific, Waltham, Mass., USA).

Sample a: Without PVP

The stained λDNA was dissolved in 0.1× Tris-Borateethylenediaminetetraaceticacid (TBE) buffer.

Sample b: With PVP

The stained λDNA was dissolved in 0.05×TBE buffer containing 0.1 w/v % polyvinyl-pyrrolidone (PVP).

In both the samples, the pH of the TBE buffer was 7.8, and the concentration of λDNA was 0.1 μg/mL.

(3) Nucleic Acid Electrophoresis

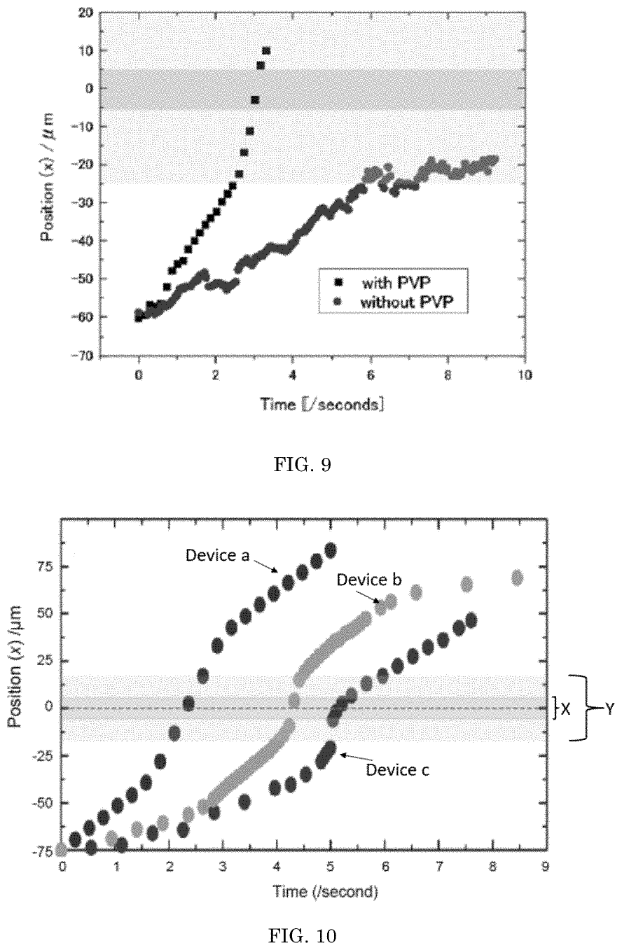

A DC voltage of 5 V was applied to the fabricated device. Motion of the nucleic acid was observed by an optical microscope. FIG. 9 indicates a measurement result. Note that “position (x)” of the vertical axis of FIG. 9 corresponds to an intermediate position of the channel corresponding to the sample measurement channel 32 at which the width is narrowest. The moving velocity of λDNA contained in the sample b with PVP was higher than λDNA contained in the sample a without PVP. This is considered to be because the PVP suppressed an electroosmotic flow (EOF) occurring in the first taper channel 33 of the device 1. It was revealed that, when the device 1 disclosed in the present application is used in the tunnel current measuring method or the nucleic acid sequence reading method, it is preferable to add a surfactant such as PVP in order to improve the reading rate of a nucleic acid.

Shape of First Taper Channel 33

### Example 7

Next, an experiment was performed to confirm influence of the shape of the first taper channel 33 on the moving velocity of a sample.

(1) Fabrication of a Device

A device was fabricated in which W1 and W1a of a device (hereafter, referred to as “Device b”) were changed as follows from the size described in Example 6. The sizes other than W1 and W1a were the same as Device b.

Device a: W1=W1=5 μm

Device c: W1=W1a=0.5 μm

(2) Adjustment of a Sample Liquid

As a sample (beads), 40 nm polystyrene florescent particles (FluoSpheres (registered trademark) Carboxylate-Modified Microspheres yellow-green fluorescent, ThermoFisher Co. Ltd., Waltham, Mass., USA) was used. The beads were suspended in 0.1×TBE buffer containing 0.1 w/v % PVP to have 2×10¹²/mL.

(3) Electrophoresis

A DC voltage of 5 V was applied to the fabricated device.

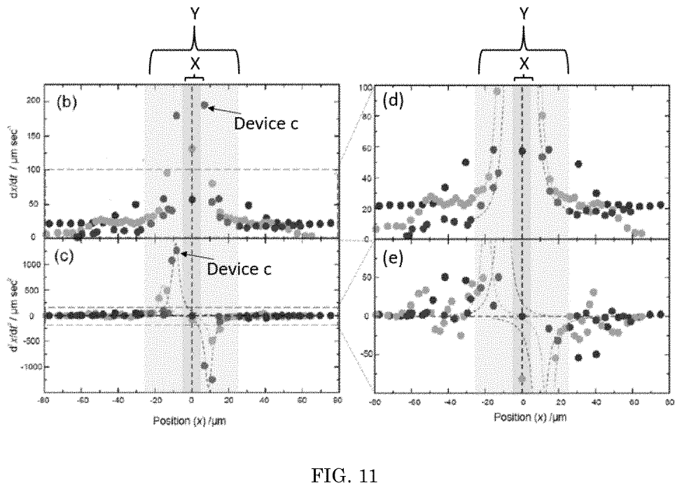

FIG. 10 indicates a measurement result. The range X in FIG. 10 represents a portion corresponding to the sample measurement channel 32 (L2 of FIG. 1A), and the range Y represents a portion corresponding to a part from the inlet of the first taper channel 33 to the outlet of the second taper channel 35 (L1+L2+L1a of FIG. 1A) (hereafter, a portion corresponding to Y may be referred to as “micro-channel portion”). Analysis illustrated in FIG. 11 was performed from the measurement result of FIG. 10. FIG. 11 (b) is a graph in which the position and the moving velocity when an individual bead of Devices a to c passes are plotted. FIG. 11 (c) is a graph in which the position and the acceleration when an individual bead of Devices a to c passes are plotted. FIG. 11 (d) is an enlarged view of FIG. 11 (b), and FIG. 11 (e) is an enlarged view of FIG. 11 (c). Note that, since FIG. 11 (b) to (e) are diagrams illustrating an overview, all the plot symbols are represented by black solid circles.

An analysis result of FIG. 11 and what was revealed are as follows.

(1) The moving velocity and the acceleration of the beads differed depending on the shape of the first taper channel 33.

(2) As illustrated in FIGS. 11 (b) and (d), although the moving velocity of the bead before and immediately after entering the micro-channel portion was stable in all the devices of Devices a to c, the moving velocity increased after fully entering the micro-channel portion. Then, the velocity decreased toward the outlet of the micro-channel portion after having a peak at Position 0. The velocities (the velocity at Position (x)) calculated from trace data on 10 beads are as follows.

Device a: 225±63 μm/s

Device b: 145±23 μm/s

Device c: 45±27 μm/s

In general, the velocity v of a sample particle is determined by the sum of an electrophoresis velocity vep and a velocity veo of an electroosmotic flow from a channel. Respective velocities, that is, vep and veo are in a proportional relationship between a DC electric field E and a velocity v and thus expressed by the following Equation.

v=vep+veo=(μep+μeo)E (1)

It was implied that a stable electric field was formed in the micro-channel portion of all the devices of Devices a to c, and it was found that stable flow control is possible.

(3) From the data indicated by FIGS. 11 (c) and (e), the maximum accelerations in the acceleration region (the left side of Position 0) of the micro-channel portion are as follows.

Device a: 52 μm/s²

Device b: 486 μm/s²

Device c: 1264 μm/s²

As is clear from the above calculation result, a larger value of W2/W1 (a larger angle of the taper of the first taper channel 33) resulted in a larger acceleration. When the acceleration is larger, the force received by the micro-channel portion becomes larger, which contributes to entropy dissociation energy of an elongate sample such as a nucleic acid and can elongate the elongate sample. Further, when the acceleration is larger, the number of samples (the number of nucleic acids) per unit time that pass through the gap G between the measuring electrodes can be increased. Further, for the same number of samples (the number of nucleic acids) that pass between the measuring electrodes per unit time, a smaller value of W2/W1 requires only a smaller value of the voltage applied for electrophoresis, and therefore measurement noise caused by the voltage applied for electrophoresis can be further reduced.

(4) As described above, it was confirmed that adjustment of the ratio of reducing the width of the first taper channel 33 from the sample supply channel 31 to the sample measurement channel 32 synergistically achieves the advantageous effects such as improvement in a reading rate of a sample and/or reduction in measurement noise caused by a voltage applied for electrophoresis, elongation of an elongate sample such as a nucleic acid, or the like.

The use of the device disclosed in the present application can reduce noise caused by the voltage applied for electrophoresis when measuring tunnel current occurring when a sample passes between the measuring electrodes 4. Therefore, the disclosed device is useful in development of analysis devices in analysis instrument industry.

### LIST OF REFERENCE SYMBOLS

1 device
2 base material
2a substrate
2b insulating layer
2c deposition layer
2d resist layer
3 channel
31 sample supply channel
32 sample measurement channel
33 first taper channel
34 sample collection channel
35 second taper channel
4, 4a, 4b measuring electrode
5 cover member
6, 6a, 6b electrophoresis power source
61 electrophoresis first electrode
62 electrophoresis second electrode
7 measuring unit
7a tunnel current detection unit
7b tunnel current measuring power source
8 analysis unit
9 display unit
10 program memory
11 control unit
100 tunnel current measuring apparatus
100a nucleic acid sequence reading apparatus

What is claimed is:

1. A device used in measurement of tunnel current, the device including:
a base material;
a channel formed in the base material; and
a pair of measuring electrodes for measuring tunnel current occurring when a sample passes between the pair of measuring electrodes,
wherein the channel includes
a sample supply channel,
a sample measurement channel in which the measuring electrodes are arranged,
a first taper channel arranged between the sample supply channel and the sample measurement channel and having a channel width that decreases from the sample supply channel to the sample measurement channel, and
a sample collection channel used for collecting a sample that passed through the sample measurement channel,
wherein the first taper channel has a shape that suppresses occurrence of an electroosmotic flow, and
wherein a width of a connection part between the first taper channel and the sample measurement channel is 20 nm to 200 nm.

2. The device according to claim 1,
wherein W2/W1 is 10 to 20, where W1 denotes the width of the connection part between the first taper channel and the sample measurement channel, and W2 denotes the width of a connection part between the first taper channel and the sample supply channel, and
wherein L1/W2 is 0.5 to 5, where L1 denotes a length between the connection part between the first taper channel and the sample measurement channel and the connection part between the first taper channel and the sample supply channel.

3. The device according to claim 1, wherein the length of the sample measurement channel is 20 nm to 1000 nm.

4. The device according to claim 2, wherein the length of the sample measurement channel is 20 nm to 1000 nm.

5. The device according to claim 1, further including a second taper channel arranged between the sample measurement channel and the sample collection channel and having a channel width that increases from the sample measurement channel to the sample collection channel.

6. The device according to claim 2, further including a second taper channel arranged between the sample measurement channel and the sample collection channel and having a channel width that increases from the sample measurement channel to the sample collection channel.

7. The device according to claim 3, further including a second taper channel arranged between the sample measurement channel and the sample collection channel and having a channel width that increases from the sample measurement channel to the sample collection channel.

8. The device according to claim 4, further including a second taper channel arranged between the sample measurement channel and the sample collection channel and having a channel width that increases from the sample measurement channel to the sample collection channel.

9. The device according to claim 1, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

10. The device according to claim 2, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

11. The device according to claim 3, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

12. The device according to claim 4, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

13. The device according to claim 5, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

14. The device according to claim 6, wherein W1=W1a, W2=W2a, and L1=L1a are satisfied, where W1a denotes a width of a connection part between the sample measurement channel and the second taper channel, W2a denotes a width of a connection part between the second taper channel and the sample collection channel, and L1a denotes a length between the connection part between the second taper channel and the sample measurement channel and the connection part between the second taper channel and the sample collection channel.

15. A tunnel current measuring apparatus including: the device according to claim 1; an electrophoresis power source; and a measuring unit,
wherein the electrophoresis power source applies a voltage of 10 mV to 5 V to an electrophoresis electrode.

16. A nucleic acid sequence reading apparatus including: the tunnel current measuring apparatus according to claim 15; and an analysis unit,
wherein the sample is a nucleic acid, and
wherein the analysis unit identifies a nucleic acid sequence from a measurement result of tunnel current acquired by the tunnel current measuring apparatus.

17. A tunnel current measuring method using a device,
wherein the device includes
a base material,
a channel formed in the base material, and
a pair of measuring electrodes for measuring tunnel current occurring when a sample passes between the pair of measuring electrodes,
wherein the channel includes
a sample supply channel,
a sample measurement channel in which the measuring electrodes are arranged,
a first taper channel arranged between the sample supply channel and the sample measurement channel and having a channel width that decreases from the sample supply channel to the sample measurement channel, and
a sample collection channel used for collecting a sample that passed through the sample measurement channel,
wherein the first taper channel has a shape that suppresses occurrence of an electroosmotic flow, and
wherein a width of a connection part between the first taper channel and the sample measurement channel is 20 nm to 200 nm,
the tunnel current measuring method including:
a sample electrophoresis step of causing electrophoresis of a sample in the sample supply channel toward the sample collection channel by applying a voltage to the sample supply channel and the sample collection channel; and
a measuring step of measuring tunnel current occurring when a sample passes through a gap between the pair of measuring electrodes arranged in the sample measurement channel.

18. The tunnel current measuring method according to claim 17, wherein in the sample electrophoresis step, a voltage of 10 mV to 5 V is applied.

19. The tunnel current measuring method according to claim 17,
wherein W2/W1 is 10 to 20, where W1 denotes the width of the connection part between the first taper channel and the sample measurement channel, and W2 denotes the width of a connection part between the first taper channel and the sample supply channel, and
wherein L1/W2 is 0.5 to 5, where L1 denotes a length between the connection part between the first taper channel and the sample measurement channel and the connection part between the first taper channel and the sample supply channel.

20. A nucleic acid sequence reading method, wherein the sample is a nucleic acid,
the method including a nucleic acid sequence reading step of identifying a nucleic acid sequence from a measurement result of tunnel current acquired by the measuring step of the tunnel current measuring method according to claim 17.
